(19) United States
(12) Patent Application Publication
(10) Pub. No.: US 2024/0182399 A1
(43) Pub. Date: Jun. 6, 2024

(54) PRODUCTION OF IRON (II) OXALATE

(71) Applicant: VSPC PTY LTD., West Perth (AU)

(72) Inventors: John WORSLEY, Wacol, Queensland (AU); Aabhash SHRESTHA, Wacol, Queensland (AU)

(21) Appl. No.: 18/284,982

(22) PCT Filed: Mar. 29, 2022
(86) PCT No.: PCT/AU2022/050283

§ 371 Date: Sep. 29, 2023

(30) Foreign Application Priority Data
Apr. 1, 2021 (AU) 2021900975

(51) Int. Cl. C07C 51/41; C07C 51/47

(52) U.S. Cl. CPC C07C 51/412; C07C 51/418; C07C 51/47

(57) ABSTRACT

Disclosed herein is a method for producing iron (II) oxalate from an insoluble iron material, the method comprising digesting the insoluble iron material with a digestion liquor comprising one or more acids, and reacting digested iron species with a source of oxalate, the oxalate being in stoichiometric excess relative to the digested iron species, to form a slurry comprising iron (II) oxalate and residual oxalate: separating the iron (II) oxalate from the slurry to provide a solids stream comprising the iron (II) oxalate and a liquids stream comprising residual oxalate; and recycling the liquids stream as the source of oxalate or a component thereof.

### FIELD

The present invention relates to a method for producing iron (II) oxalate.

### BACKGROUND

Iron (II) oxalate is an important commodity and finds use in the production of electronic components and, in particular, as an intermediate in the production of lithium iron phosphate (LFP) such as for use as a cathode material in lithium-ion batteries. However, for these applications a high purity iron (II) oxalate is required. Current processes for producing this iron (II) oxalate are relatively costly.

It is desirable to provide new processes for the bulk production of iron (II) oxalate that can produce high quality iron (II) oxalate with reduced cost.

It is an object of the invention to address at least one shortcoming of the prior art and/or provide a useful alternative.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF INVENTION

In a first aspect of the invention, there is provided a method for producing iron (II) oxalate from an insoluble iron material, the method comprising:
digesting the insoluble iron material with a digestion liquor comprising one or more acids, and reacting digested iron species with a source of oxalate, the oxalate being in stoichiometric excess relative to the digested iron species, to form a slurry comprising iron (II) oxalate and residual oxalate;
separating the iron (II) oxalate from the slurry to provide a solids stream comprising the iron (II) oxalate and a liquids stream comprising residual oxalate; and
recycling the liquids stream as the source of oxalate or a component thereof.

This method is particularly applicable to producing high purity iron (II) oxalate from a high-quality iron feed material, such as an iron metal feed or synthetically produced iron compounds which is substantially free of impurities. By substantially free of impurities, it is meant that the insoluble iron material consists of or consists essentially of iron or iron compounds and incidental impurities. By way of example, in a non-limiting disclosure the incidental impurities comprise one or more of: 0.1 wt % Al or less, 0.15 wt % Ca or less, 0.15 wt % Mg or less, 0.03 wt % Na or less, 0.15 wt % K or less, 10 ppm Cr or less, Ti, 1500 ppm Mn or less, 100 ppm P or less, 25 ppm Sr or less, and 50 ppm Ba or less.

In an embodiment, the method produces an iron (II) oxalate product with an Fe content of at least 29.7 wt %.

Notwithstanding the above, the skilled person will appreciate that this method can also be used to produce lower purity iron (II) oxalate from low quality iron feed material sources, such as scrap iron or iron compounds comprising impurities, or low-quality feed materials such as iron ore, iron ore concentrates, iron tailings and the like. However, certain embodiments of the invention as described herein provide methods for recovering high purity iron (II) oxalate from low-quality iron feed materials.

In an embodiment, the source of oxalate is oxalic acid.

In one form of this embodiment, the digestion liquor comprises, consists of, or consists essentially of an aqueous solution of oxalic acid. The inventors have found that oxalic acid is able to digest iron feed materials, particularly magnetite and goethite, with the oxalate anion subsequently reacting with the digested iron species to form iron (II) oxalate.

In alternative forms of this embodiment, oxalic acid is added to the digestion liquor to provide the source of oxalate during or after the digesting step.

In forms of the invention in which the source of oxalate is oxalic acid, the step of recycling the liquids stream as the source of oxalate or a component thereof, further comprises recycling the liquids stream as digestion liquor.

In an embodiment, the digestion liquor comprises, consists of, or consists essentially of an aqueous solution of a mineral acid. In one form of this embodiment, an aqueous solution of oxalic acid is added to the digestion liquor to provide the source of oxalate during or after the digesting step.

In another embodiment, the digestion liquor comprises, consists of, or consists essentially of an aqueous solution of mineral acid and oxalic acid, the oxalic acid providing the source of oxalate.

In embodiments in which the digestion liquor comprises a mineral acid and the source of oxalate is oxalic acid, the step of reacting digested iron with the source of oxalate regenerates the mineral acid. In such embodiments, the method comprises:
contacting the insoluble iron material with the digestion liquor, the digestion liquor comprising the mineral acid, and digesting the insoluble iron material to form a reaction solution comprising a soluble iron compound;
reacting the soluble iron compound in the reaction solution with oxalic acid to form a slurry comprising iron (II) oxalate, residual oxalic acid, and regenerated mineral acid;
separating the iron (II) oxalate from the slurry to provide a solids stream comprising the iron (II) oxalate and a liquids stream comprising residual oxalic acid and the regenerated mineral acid; and
recycling the liquids stream as digestion liquor.

In an embodiment, the insoluble iron feed material comprises, consists of, or consists essentially of an insoluble iron compound reactive with the digestion liquor to form the soluble iron compound.

In an embodiment, the insoluble iron feed material comprises, consists of, or consists essentially of iron metal and/or synthetically produced iron compounds. The iron metal may be, for example, in the form of powdered iron, granulated iron, iron filings, iron particulates, otherwise comminuted iron, and/or iron containing waste. The iron compounds may be selected from the group consisting of iron oxide, iron oxide-hydroxide, and/or iron carbonate. As mentioned previously, the provision of high-quality iron or iron compounds results in the formation of high purity iron (II) oxalate. However, if impurities or contaminants are present, then in the absence of additional processing steps, a lower purity iron (II) oxalate is produced.

In an embodiment, the insoluble iron feed material is provided in the form of tailings from mineral processing operations that comprise iron compounds (such as one or more of the iron compounds listed above) and/or an iron ore and/or an iron ore concentrate. In cases where the insoluble iron feed material is an iron ore or an iron ore concentrate, the insoluble iron feed material is preferably selected from the group consisting of: haematite, magnetite, goethite, limonite, and/or siderite.

The inventors have found that the digestion of iron tailings, iron ores, and iron ore concentrates and subsequent reaction of digested iron with oxalate can result in a mixture of iron (II) oxalate and iron (III) oxalate. In such cases, the method further comprises reducing iron (III) oxalate formed during the reaction to iron (II) oxalate. A variety of reduction methods are contemplated, e.g. by generally contacting the iron (III) oxalate with a reducing source (e.g. a chemical source, a source of radiation such as light or UV, an electrical source such as via electrolysis etc.). Preferably the step of reducing the iron (III) oxalate comprises contacting the slurry with a source of iron to reduce iron (III) oxalate formed during reaction to iron (II) oxalate.

Tailings, iron ores, and iron ore concentrates are low quality sources of iron feed material and typically include impurities and/or contaminants. Whilst the method of the invention can be applied to produce iron (II) oxalate, the resultant iron (II) oxalate can be of low quality.

The inventors have devised additional processing steps that are able to produce a high purity iron (II) oxalate from a low-quality iron feed material. In particular, in an embodiment, prior to the step of separating the iron (II) oxalate from the slurry to provide the solids stream and the liquids stream, the method further comprises:
oxidising iron (II) oxalate in the slurry to form a reaction solution comprising soluble iron (III) oxalate and solid impurities;
removing solid impurities from the reaction solution; and
reducing the iron (III) oxalate to form a slurry comprising precipitated iron (II) oxalate.

As discussed above, the skilled person will appreciate that the iron (III) oxalate may be reduced using a variety of approaches. However, it is preferred that the step of reducing the iron (III) oxalate comprises contacting the reaction solution with a source of iron to reduce the iron (III) oxalate and form a slurry comprising precipitated iron (II) oxalate.

These additional processing steps can be applied to remove impurities having low solubility in the digestion liquor (which can be varied to limit solubility of impurities) or which remain in solution when the dissolved iron is precipitated as iron (II) oxalate, and that through varying the process conditions can be optimised to minimise impurities in the product iron (II) oxalate. By way of example, a non-limiting disclosure of solid impurities includes one or more of gangue and/or one or more mineral impurities selected from the group consisting of Si, Al, Ca, Mg, Mn, Na, K, Cr, Ti, Mn, P. Sr, and Ba contaminant compounds.

It is preferred that the iron (II) oxalate is oxidised to iron (III) oxalate with an oxidant. A suitable oxidant is hydrogen peroxide.

In forms of the invention where the insoluble iron material comprises tailings and/or iron ore and/or iron ore concentrates, the inventors have found that addition of iron metal to the insoluble iron material (such as in the form of comminuted iron discussed previously) accelerates digestion of tailings/iron ore/iron ore concentrates. As such, it is preferred that the insoluble iron material further comprises iron metal. More preferably, the insoluble iron material further comprises iron metal at up to 25 wt % of the total weight of the insoluble iron material.

In a second aspect of the invention, there is provided a method for producing iron (II) oxalate from an iron material comprising impurities, the method comprising:
contacting the insoluble iron material with a digestion liquor comprising one or more acids, and reacting digested iron species with a source of oxalate, the oxalate being in stoichiometric excess relative to the digested iron species, to form a slurry comprising iron (II) oxalate, residual oxalate, and solid impurities;
oxidizing the iron (II) oxalate in the slurry to form a reaction solution, the reaction solution comprising soluble iron (III) oxalate, residual oxalate, and solid impurities;
separating the solid impurities from the reaction solution;
reducing soluble iron (III) oxalate and forming a slurry comprising iron (II) oxalate and residual oxalate;
separating the iron (II) oxalate from the slurry to provide a solids stream comprising the iron (II) oxalate and a liquid stream comprising residual oxalate; and
recycling the liquids stream as the source of oxalate or a component thereof.

In an embodiment, the step of reducing the soluble iron (III) oxalate comprises contacting the reaction solution with a source of iron to reduce the soluble iron (III) oxalate and form the slurry comprising iron (II) oxalate.

In an embodiment, the source of oxalate is oxalic acid.

In one form of this embodiment, the digestion liquor comprises, consists of, or consists essentially of an aqueous solution of oxalic acid. As previously discussed, this embodiment is particularly well suited to magnetite and goethite.

In alternative forms of this embodiment, oxalic acid is added to the digestion liquor to provide the source of oxalate during or after the digesting step. The oxalic acid may be added in solid form, e.g. as granules or, for example, in an aqueous solution.

In forms of the invention in which the source of oxalate is oxalic acid, the step of recycling the liquids stream as the source of oxalate or a component thereof, further comprises recycling the liquids stream as digestion liquor.

In an embodiment, the digestion liquor comprises, consists of, or consists essentially of an aqueous solution of a mineral acid. In one form of this embodiment, an aqueous solution of oxalic acid is added to the digestion liquor to provide the source of oxalate during or after the digesting step.

In another embodiment, the digestion liquor comprises, consists of, or consists essentially of an aqueous solution of mineral acid and oxalic acid, the oxalic acid providing the source of oxalate.

In embodiments in which the digestion liquor comprises a mineral acid and the source of oxalate is oxalic acid, the step of reacting digested iron with the source of oxalate regenerates the mineral acid. In such embodiments, the method comprises:
contacting the insoluble iron material with the digestion liquor comprising a mineral acid and digesting the insoluble iron material to form a soluble iron compound;
reacting the soluble iron compound with oxalic acid to form the slurry comprising iron (II) oxalate, residual oxalic acid, regenerated mineral acid, and solid impurities;
oxidizing the iron (II) oxalate in the slurry to form the reaction solution, the reaction solution comprising soluble iron (III) oxalate, residual oxalic acid, regenerated mineral acid, and solid impurities;
separating the solid impurities from the reaction solution;
reducing soluble iron (III) oxalate and forming the slurry, the slurry comprising iron (II) oxalate, residual oxalic acid, and regenerated mineral acid;
separating the iron (II) oxalate from the slurry to provide the solids stream comprising the iron (II) oxalate and the liquid stream comprising the residual oxalic acid and the regenerated mineral acid; and
recycling the recovered liquids stream as digestion liquor.

As discussed above, the second aspect of the invention provides a method for producing high purity iron (II) oxalate from impure iron feed materials, such as those that comprise impurities. The inventors have found that these impurities either remain as undigested solids, or if digested, form insoluble oxalates (which unlike iron do not oxidise to, or are not readily oxidised to, soluble oxalates). Thus, the method according to the second aspect of the invention is particularly well suited to low quality iron feedstocks such as tailings from mineral processing operations comprising one or more iron compounds and/or an iron ore and/or an iron ore concentrate.

As discussed above, the invention can be applied to remove impurities having low solubility in the digestion liquor (which can be varied to limit solubility of impurities) or which remain in solution when the dissolved iron is precipitated as iron (II) oxalate, and that through varying the process conditions can be optimised to minimise impurities in the product iron (II) oxalate. By way of example, a non-limiting disclosure of solid impurities includes one or more of gangue and/or one or more mineral impurities selected from the group consisting of Si, Al, Ca, Mg, Mn, Na, K, Cr, Ti, Mn, P, Sr, and Ba contaminant compounds.

In an embodiment, the method produces an iron (II) oxalate product with an Fe content of at least 29.7 wt %.

In view of the above, in a preferred embodiment, the insoluble iron feed material is provided in the form of tailings from mineral processing operations that comprise iron compounds (such as one or more of the iron compounds listed previously) and/or an iron ore and/or an iron ore concentrate. In cases where the insoluble iron feed material is an iron ore or an iron ore concentrate, the insoluble iron feed material is preferably selected from the group consisting of: haematite, magnetite, goethite, limonite, and/or siderite.

In one form of the above embodiment, the insoluble iron feed material further comprises iron metal (such as in the form of comminuted iron discussed previously) to accelerate digestion. This iron metal may likewise comprise solid impurities, which will be removed during the step of separating the solid impurities from the reaction solution.

In an embodiment, the step oxidizing the iron (II) oxalate includes reacting the iron (II) oxalate with an oxidant. A suitable oxidant is hydrogen peroxide.

With reference to embodiments of the first and second aspects which employ a mineral acid, the skilled person will appreciate that a range of mineral acids may be used, provided that these are suitable to digest iron and form a soluble iron compound. Suitable mineral acids include H₂SO₄, HCl, or mixtures thereof. It is preferred that the mineral acid is H₂SO₄. In such cases, the soluble iron compound is iron (II) sulphate.

In embodiments of the first and second aspects, the insoluble iron feed material is digested at a temperature of from about 50° C. to about 100° C. and/or the soluble iron compound is reacted with a source of oxalate at a temperature of from about 50° ° C. to about 100° C. Preferably the temperature is from about 80° C. More preferably the temperature is from about 85° C. Even more preferably the temperature is from about 90° C. Most preferably the temperature is from about 95° C.

In embodiments of the first and second aspects, the insoluble iron feed material is digested substantially under atmospheric pressure and/or the soluble iron compound is reacted with the source of oxalate substantially under atmospheric pressure.

In embodiments of the first and second aspects, the molar ratio of oxalic acid to iron in the insoluble iron feed material is at least 1.5. More preferably, the ratio is about 2. Most preferably, the ratio is at least or up to 3.

In embodiments of the first and second aspects, the digestion liquor comprises regenerated mineral acid recovered from reaction between a soluble iron compound and oxalic acid.

In embodiments of the first and second aspects, the digestion liquor comprises residual soluble iron compounds and/or oxalic acid.

In embodiments of the first and second aspects, the separating step(s) comprise a solid-liquid separation step. Any suitable separation process known to be suitable may be used, including filtration, thickening, centrifugation, settling, cyclone separation, hydrocyclone separation, or the like.

In a third aspect of the invention there is provided a process for producing iron (II) oxalate, the process comprising:
providing a feed of an insoluble iron material into a reactor;
providing a feed of a digestion liquor comprising one or more acids into the reactor;
contacting the insoluble iron material with the digestion liquor in the reactor to digest the insoluble iron feed material;
reacting digested iron species in the reactor with a source of oxalate, the oxalate being in stoichiometric excess relative to the digested iron species, to form a slurry comprising iron (II) oxalate and residual oxalate;
subjecting the slurry to a solid-liquid separation process to provide a solids stream comprising iron (II) oxalate and a liquids stream comprising residual oxalate;
recycling the liquids stream into the reactor as the source of oxalate or a component thereof.

As with the first aspect of the invention, the third aspect is particularly applicable to producing high purity iron (II) oxalate from a high-quality iron feed material, such as an iron metal feed or synthetically produced iron compounds which is substantially free of impurities. By substantially free of impurities, it is meant that the insoluble iron material consists of or consists essentially of iron or iron compounds and incidental impurities. By way of example, in a non-limiting disclosure the incidental impurities comprise one or more of: 0.1 wt % Al or less, 0.15 wt % Ca or less, 0.15 wt % Mg or less, 0.03 wt % Na or less, 0.15 wt % K or less, 10 ppm Cr or less, Ti, 1500 ppm Mn or less, 100 ppm P or less, 25 ppm Sr or less, and 50 ppm Ba or less.

In an embodiment, the process produces an iron (II) oxalate product with an Fe content of at least 29.7 wt %.

Notwithstanding the above, as with the first aspect of the invention, the skilled person will appreciate that this method can also be used to produce lower purity iron (II) oxalate from low quality iron feed material sources.

In an embodiment, the source of oxalate is oxalic acid.

In one form of this embodiment, the digestion liquor comprises, consists of, or consists essentially of an aqueous solution of oxalic acid. As previously discussed, this embodiment is particularly well suited to magnetite and goethite.

In alternative forms of this embodiment, oxalic acid is added to the digestion liquor to provide the source of oxalate during or after the digesting step. The oxalic acid may be added in solid form e.g. as granules, or in the form of an aqueous solution.

In forms of the invention in which the source of oxalate is oxalic acid, the step of recycling the liquids stream as the source of oxalate or a component thereof, further comprises recycling the liquids stream as digestion liquor.

In an embodiment, the digestion liquor comprises, consists of, or consists essentially of an aqueous solution of a mineral acid. In one form of this embodiment, oxalic acid is added to the digestion liquor to provide the source of oxalate during or after the digesting step.

In another embodiment, the digestion liquor comprises, consists or, or consists essentially of an aqueous solution of mineral acid and oxalic acid, the oxalic acid providing the source of oxalate.

In embodiments in which the digestion liquor comprises a mineral acid and the source of oxalate is oxalic acid, the step of reacting digested iron with the source of oxalate regenerates the mineral acid. In such embodiments, the process comprises:
contacting the insoluble iron material with the digestion liquor in the reactor to digest the insoluble iron material, the digestion liquor comprising a mineral acid to digest the insoluble iron material and form a reaction solution comprising a soluble iron compound;
reacting the soluble iron compound in the reaction solution with oxalic acid in the reaction vessel to form the slurry, the slurry comprising iron (II) oxalate, residual oxalic acid, and regenerated mineral acid;
subjecting the slurry to the solid-liquid separation process to provide the solids stream comprising the iron (II) oxalate and the liquids stream, the liquids stream comprising residual oxalic acid and the regenerated mineral acid;
recycling the liquids stream comprising residual oxalic acid and the regenerated mineral acid into the reactor as digestion liquor (which may be recycled to the reactor as a component of the feed of the digestion liquor or as a separate recycle stream feed of digestion liquor).

In an embodiment, the insoluble iron feed material comprises iron metal. The iron metal may be, for example, in the form of powdered iron, granulated iron, iron filings, iron particulates, or otherwise comminuted iron.

In an embodiment, the insoluble iron feed material comprises, consists of, or consists essentially of an iron compound (preferably an insoluble iron compound) reactive with the mineral acid to form the soluble iron compound.

In one form of the above embodiment, the insoluble iron feed material is provided in the form of tailings from mineral processing operations that comprise the iron compound.

In one form of the above embodiment, the iron compound is in the form of an iron ore, iron ore concentrate, or synthetically produced iron compound, the iron compound selected from the group consisting of iron oxide, iron oxide-hydroxide, and/or iron carbonate. In cases where the insoluble iron feed material is an iron ore or an iron ore concentrate, the insoluble iron feed material is preferably selected from the group consisting of: haematite, magnetite, goethite, limonite, and/or siderite.

In one form of the above embodiment, the method further comprises reducing iron (III) oxalate formed during the reaction to iron (II) oxalate. Preferably, the step of reducing iron (III) oxalate comprises contacting the reaction solution with a source of iron (e.g. by adding iron to the slurry) to reduce iron (III) oxalate formed during reaction to iron (II) oxalate.

In embodiments in which the feed of the insoluble iron material comprises impurities (as generally discussed above in relation to the first and second aspects) and it is desired to remove these impurities, the process further comprises the steps of:
oxidising iron (II) oxalate in the slurry to form a reaction solution comprising soluble iron (III) oxalate and solid impurities;
removing the solid impurities from the reaction solution; and
reducing iron (III) oxalate formed during the reaction to iron (II) oxalate.

Preferably, the step of reducing iron (III) oxalate comprises contacting-the reaction solution with a source of iron to reduce the iron (III) oxalate and form a slurry comprising iron (II) oxalate.

As discussed previously, a non-limiting disclosure of solid impurities includes one or more of gangue and/or one or more mineral impurities selected from the group consisting of Si, Al, Ca, Mg, Mn, Na, K, Cr, Ti, Mn, P, Sr, and Ba contaminant compounds.

It is preferred that the iron (II) oxalate is oxidised to iron (III) oxalate with an oxidant. A suitable oxidant is hydrogen peroxide.

In a fourth aspect of the invention there is provided a process for producing iron (II) oxalate from an iron feed material comprising impurities, the process comprising:
providing a feed of an insoluble iron material comprising impurities into a reactor;
providing a feed of a digestion liquor comprising one or more acids into the reactor;
contacting the insoluble iron material with the digestion liquor in the reactor to digest the insoluble iron feed material;
reacting digested iron species in the reactor with a source of oxalate, the oxalate being in stoichiometric excess relative to the digested iron species, to form a slurry comprising iron (II) oxalate, residual oxalate, and solid impurities;
oxidizing the iron (II) oxalate in the slurry to form a reaction solution, the reaction solution comprising soluble iron (III) oxalate, residual oxalate, and solid impurities;
subjecting the reaction solution to a first solid-liquid separation process to separate the solid impurities from the reaction solution;
reducing the soluble iron (III) oxalate and forming a slurry comprising iron (II) oxalate and residual oxalate;
subjecting the slurry to a second solid-liquid separation process to provide a solids stream comprising iron (II) oxalate and a liquids stream comprising residual oxalate;
recycling the liquids stream into the reactor as the source of oxalate or a component thereof.

In an embodiment, the step of reducing the soluble iron (III) oxalate comprises contacting the reaction solution with a source of iron to reduce the soluble iron (III) oxalate to form the slurry comprising iron (II) oxalate.

In an embodiment, the source of oxalate is oxalic acid.

In one form of this embodiment, the digestion liquor comprises, consists of, or consists essentially of an aqueous solution of oxalic acid. As previously discussed, this embodiment is particularly well suited to magnetite and goethite.

In alternative forms of this embodiment, oxalic acid is added to the digestion liquor to provide the source of oxalate during or after the digesting step.

In forms of the invention in which the source of oxalate is oxalic acid, the step of recycling the liquids stream as the source of oxalate or a component thereof, further comprises recycling the liquids stream as digestion liquor.

In an embodiment, the digestion liquor comprises, consists of, or consists essentially of an aqueous solution of a mineral acid. In one form of this embodiment, an aqueous solution of oxalic acid is added to the digestion liquor to provide the source of oxalate during or after the digesting step.

In another embodiment, the digestion liquor comprises, consists or, or consists essentially of an aqueous solution of mineral acid and oxalic acid, the oxalic acid providing the source of oxalate.

In embodiments in which the digestion liquor comprises a mineral acid and the source of oxalate is oxalic acid, the step of reacting digested iron with the source of oxalate regenerates the mineral acid. In such embodiments, the process comprises:
contacting the insoluble iron material with the digestion liquor in the reactor to digest the insoluble iron material, the digestion liquor comprising a mineral acid to digest the insoluble iron material and form a reaction solution comprising a soluble iron compound;
reacting the soluble iron compound in the reaction solution with oxalic acid in the reactor to form the slurry, the slurry comprising iron (II) oxalate, residual oxalic acid, and regenerated mineral acid;
oxidising iron (II) oxalate in the slurry to form a reaction solution comprising soluble iron (III) oxalate and solid impurities subjecting the reaction solution to the first solid-liquid separation process to separate the solid impurities from the reaction solution;
reducing the soluble iron (III) oxalate and forming the slurry comprising iron (II) oxalate, residual oxalic acid, and regenerated mineral acid;
subjecting the slurry to the second solid-liquid separation process to provide the solids stream comprising the iron (II) oxalate and the liquids stream, the liquid stream comprising residual oxalic acid and regenerated mineral acid;
recycling the liquids stream comprising the residual oxalic acid and the regenerated mineral acid into the reactor as digestion liquor (which may be recycled to the reactor as a component of the feed of the digestion liquor or as a separate recycle stream feed of digestion liquor).

The fourth aspect of the invention provides a process for producing high purity iron (II) oxalate from impure iron feed materials, such as those that comprise impurities. As discussed previously, a non-limiting disclosure of solid impurities includes one or more of gangue and/or one or more mineral impurities selected from the group consisting of Si, Al, Ca, Mg, Mn, Na, K, Cr, Ti, Mn, P, Sr, and Ba contaminant compounds.

In an embodiment, the method produces an iron (II) oxalate product with an Fe content of at least 29.7 wt %.

In embodiments of the third and fourth aspects, the insoluble iron feed material comprises, consists of, or consists essentially of an iron compound (preferably an insoluble iron compound) reactive with the mineral acid to form the soluble iron compound.

In embodiments of the third and fourth aspects, the insoluble iron feed material is provided in the form of tailings from mineral processing operations that comprise the iron compound.

In embodiments of the third and fourth aspects, the iron compound is in the form of an iron ore, iron ore concentrate, or synthetically produced iron compound, the iron compound selected from the group consisting of iron oxide, iron oxide-hydroxide, and/or iron carbonate. In cases where the insoluble iron feed material is an iron ore or an iron ore concentrate, the insoluble iron feed material is preferably selected from the group consisting of: haematite, magnetite, goethite, limonite, and/or siderite.

In embodiments of the third and fourth aspects, the insoluble iron feed material further comprises iron (such as in the form of comminuted iron discussed previously).

In embodiments of the third and fourth aspects in which the iron (II) oxalate is oxidized to iron (III) oxalate, the step oxidizing the iron (II) oxalate includes reacting the iron (II) oxalate with an oxidant. A suitable oxidant is hydrogen peroxide.

In embodiments of the third or fourth aspects, the mineral acids include H₂SO₄, HCl, or mixtures thereof. It is preferred that the mineral acid is H₂SO₄.

In embodiments of the third or fourth aspects, the molar ratio of oxalic acid to iron in the insoluble iron feed material is at least 1.5. More preferably, the ratio is about 2. Most preferably, the ratio is at least or up to 3.

In embodiments of the third or fourth aspects, the liquids stream comprises residual soluble iron compounds.

In embodiments of the third or fourth aspects, the reaction vessel is operated at an operating temperature of from about 50° C. to about 100° C. Preferably the temperature is from about 80° C. More preferably the temperature is from about 85° C. Even more preferably the temperature is from about 90° ° C. Most preferably the temperature is from about 95° C.

In embodiments of the third or fourth aspects, the reaction vessel is operated substantially at atmospheric pressure.

In embodiments of the third and fourth aspects, the solid-liquid separation process is selected from filtration, thickening, centrifugation, settling, cyclone separation, hydrocyclone separation, or the like.

In embodiments of the first, second, third, and fourth aspects, at least a portion of the recycled liquid stream is bled off as a purge stream, such as to remove impurities from the recycled liquid stream with the purge stream. In alternative embodiments, the liquids stream is treated to remove one or more impurities prior to recycling.

In embodiments of the first, second, third, and fourth aspects, the methods or processes further comprise subjecting the solid stream comprising the iron (II) oxalate to one or more wash steps (such as with high purity water e.g. deionized, distilled, or otherwise purified) to remove residual acid and/or soluble impurities. In one form of this embodiment, a spent wash liquor from a first wash step is recycled with the liquids stream and/or as a component of the digestion liquor, e.g. to reduce loss of any oxalate and/or acid. However, in alternative forms of this embodiment, the spent wash liquor from a first wash step is disposed of. Spent wash liquor from any subsequent steps can be disposed of. Subsequent to washing the solid stream comprising the iron (II) oxalate, the method and/or process further comprises drying the iron (II) oxalate.

In embodiments of the first, second, third, and fourth aspects, the acid to iron molar ratio is higher than 1.1.

In embodiments of the first, second, third, and fourth aspects in which oxalic acid and mineral acid are present, it is preferred that the ratio of oxalic acid to mineral acid is from about 100:0 to about 1:1.

In one or more embodiments of the first, second, third, or fourth aspects, the slurry further comprises residual iron metal (e.g. such as in the form of iron powder). In such embodiments, the method further comprises separating iron metal from the slurry. This separation may be achieved by chemical or physical separation processes. However, preferably magnetic separation is employed. It is preferred that the step of separating iron metal from the slurry is carried out prior to the step of separating the iron (II) oxalate from the slurry.

In a fifth aspect of the invention, there is provided an iron (II) oxalate product formed according to the method of the first aspect of the invention and/or embodiments and/or forms thereof, the second aspect of the invention and/or embodiments and/or forms thereof, the process of the third aspect of the invention and/or embodiments and/or forms thereof, or the process of the fourth aspect of the invention and/or embodiments and/or forms thereof.

In an embodiment, the iron (II) oxalate product has an Fe content of at least 29.7 wt %.

Further aspects of the present invention and further embodiments of the aspects described in the preceding paragraphs will become apparent from the following description, given by way of example and with reference to the accompanying drawings.

### BRIEF DESCRIPTION OF DRAWINGS

FIG. 1: Process flow diagram illustrating an embodiment of the invention for preparing iron (II) oxalate using iron powder as the raw material for sulphuric acid regeneration.

FIG. 2: Process flow diagram illustrating an embodiment of the invention for preparing iron (II) oxalate using pure iron oxide as the raw material for sulphuric acid regeneration.

FIG. 3: Process flow diagram illustrating an embodiment of the invention for preparing iron (II) oxalate using impure iron ores as the raw material for sulphuric acid regeneration.

FIG. 4: XRD spectra of lanthanide tailings and undigested residue from the tailings after the first step purification as per Example 4. The reference spectra are of Goethite. Upper spectrum represents the lanthanide tailings and lower spectrum represents the tailings residue.

FIG. 5: Process flow diagram illustrating an embodiment of the invention for preparing iron (II) oxalate from lanthanide tailings using oxalic acid as the digestion liquor as per Example 4.

### DESCRIPTION OF EMBODIMENTS

The invention broadly relates to a method of producing iron (II) oxalate that comprises acid digestion of an insoluble iron feed material to form an aqueous soluble iron compound (particularly an iron (II) compound) with an acid, and concurrently reacting that soluble iron compound with a source of oxalate that is in stoichiometric excess relative to the iron.

It is generally preferred that the insoluble iron feed material has been subjected to a size reduction process and is generally in the form of particulates, powders, filings, and/or shavings, to enhance the rate of acid digestion.

The reaction of the aqueous soluble iron compound with the source of oxalate forms iron (II) oxalate. The iron (II) oxalate, being poorly soluble in water, precipitates from solution and can be separated from the solution via a solid-liquid separation process. Once the iron (II) oxalate has been recovered from the solution, the solution including residual oxalate, can be recycled for use in subsequent conversion of iron to iron (II) oxalate.

The inventors have found that oxalic acid may beneficially be used as both a digestion reagent and as the source of oxalate. In this way, iron containing materials with readily accessible forms of iron (e.g. goethite and/or magnetite), can be easily converted to iron (II) oxalate. However, with other materials, it is desirable that the digestion liquor comprises a mineral acid, such as sulfuric acid and/or hydrochloric acid. In such cases, the inventors have found that the reaction between the digested iron species and source of oxalic acid regenerates the mineral acid. Thus, the mineral acid and/or residual oxalic acid can be recycled for use as digestion liquor and/or source of oxalate. Advantageously, this mitigates the waste treatment cost of the mineral acid making the process more cost effective and sustainable. Furthermore, the recycle stream may also comprise residual soluble iron species which may be subsequently recovered as iron (II) oxalate in future passes.

The inventors have also found that it is advantageous that the method includes contacting the insoluble iron feed material with a digestion liquor that comprises both the mineral acid and the oxalic acid to simplify the process and to enhance the digestion process through continuous regeneration of the acid as the solubilised iron compounds are converted to iron (II) oxalate.

The digestion of iron compounds and the subsequent reaction with oxalate (which is preferably in the form of oxalic acid) can form a mixture of iron (II) oxalate and iron (III) oxalate, particularly where excess oxalate is used. It should be noted that the use of a stoichiometric excess of oxalic acid is useful since this results in enhanced regeneration and subsequent recovery of the acid consumed during digestion. In such case, it is desirable to subsequently reduce the soluble iron (III) to iron (II) oxalate to improve the overall recovery efficiency of the process. A variety of approaches can be applied to reduce the iron (III) oxalate to iron (II) oxalate, e.g. chemical reduction, UV reduction, electrolysis etc. Notwithstanding these approaches, the inventors have found that addition of comminuted iron (preferably powdered iron) can reduce the iron (III) oxalate to iron (II) oxalate. The comminuted iron is first digested by the regenerated acid, and then subsequently reduces the iron (III) oxalate to iron (II) oxalate and in this process is itself precipitated as iron (II) oxalate. The regenerated acid that is consumed during the digestion of the comminuted iron is again regenerated during this reaction.

While the invention has been broadly described above with reference to iron as a feed material, a variety of different insoluble iron feed materials may be used. For example, the insoluble iron feed material can be an iron containing waste, iron ores, iron ore concentrates, or synthetically produced iron compounds. The ores/concentrates/compounds are preferably in the form of oxides, oxide-hydroxides, and carbonates.

An advantage of using a high-quality iron feed stock is that the resultant iron (II) oxalate is of high purity. However, high-quality iron feed stocks are expensive. Embodiments of the method and process of the invention are also able to make use of low-quality iron feedstocks, such as scrap iron/iron ores/iron ore concentrates/iron containing tailings. The use of these feed materials provides the method with increased flexibility and can lower the overall cost of the process compared with using high-quality iron as the feed stock.

The use of iron containing tailings and ores is particularly advantageous since these materials are abundant and low cost. However, as noted above, iron ores/concentrates include solid impurities, for example gangue, and other metals or metalloids. These solid impurities typically remain as undigested solids, and since iron (II) oxalate form as a precipitate, these solid impurities cannot be easily separated from the iron (II) oxalate.

To address this, the inventors have further devised a process whereby the iron (II) oxalates are oxidised to soluble iron (III) oxalates, e.g. through the addition of an oxidant such as hydrogen peroxide. Since the iron species are now dissolved in solution, any solid impurities (e.g. undigested solid impurities or solid impurities formed as a result of reaction with the source of oxalate) can be removed via standard solid-liquid separation processes. After removal of these impurities, the iron (III) oxalates may be reduced to iron (II) oxalates, for example, by the approaches generally discussed previously but preferably via addition of comminuted iron. The iron (II) oxalates can then be recovered in highly pure form as a solids stream with the liquid stream containing residual oxalate and/or regenerated acid being recycled for future passes. Any soluble impurities also remain in the liquid stream thus being separated from the iron (II) oxalate but being recycled. To prevent accumulation of soluble impurities in the recycle stream, a portion of the recycle stream can be bled off.

In view of the above, the invention provides methods and/or process for producing a high purity iron (II) oxalate product from iron feed stocks of varying quality.

The invention will now be discussed in relation to embodiments thereof exemplified below in which the acid is sulphuric acid.

### Example 1: Method of Producing Iron (II) Oxalate from Iron Powder

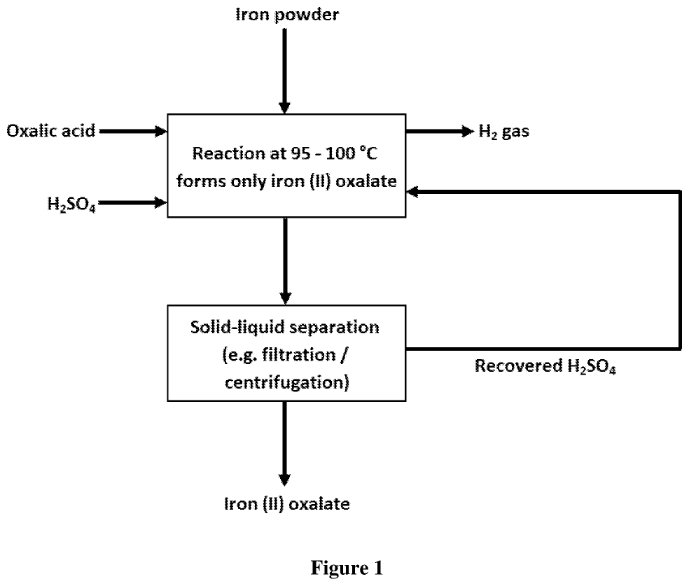

The preparation of iron (II) oxalate using iron (II) sulphate and oxalic acid generates sulphuric acid as a waste product. The cost of treating waste sulphuric acid can be mitigated if the waste acid can be effectively reutilised. FIG. 1 is a process flow diagram illustrating the reutilisation of sulphuric acid according to an embodiment of the invention and using iron powder as the iron source (or other low-cost iron such as scrap iron or sponge iron). Here, the iron powder is reacted in a mixture of sulphuric acid and oxalic acid solution.

The sulphuric acid first reacts with iron powder to form iron (II) sulphate (Equation 1). The generated iron (II) sulphate can then react with oxalic acid in the solution to produce iron (II) oxalate, which regenerates sulphuric acid (Equation 2). Iron (II) oxalate can be separated via filtration or centrifugation and the regenerated sulphuric acid can be recycled for iron dissolution. This can potentially mitigate the waste treatment cost of sulphuric acid and can make the process more sustainable.

Fe+H₂SO₄=FeSO₄+H₂↑ Equation 1

FeSO₄+C₂H₂O₄=H₂SO₄+FeC₂O₄θ& Equation 2

To validate the technical feasibility of sulphuric acid re-utilisation, four cycles of sulphuric acid regeneration were conducted. Here, the iron powder (Rapid rust) was dissolved in a mixture of sulphuric acid and oxalic acid to demonstrate the technical feasibility. Table 1 below shows that the yield of iron (II) oxalate increased with increasing the number of cycles of synthesis and it can reach 100% of the theoretical yield after the 3^{rd} cycle of regeneration. This is mainly due to some residual iron sulphate and oxalic acid in the waste sulphuric acid solution.

TABLE 1
Yield of iron (II) oxalate at different
cycles of sulphuric acid regeneration

| | Theoretical yield (g) | | | | |
|---|---|---|---|---|---|
| | 85.45 | | | | |
| | 1st cycle | 2nd cycle | 3rd cycle | 4th cycle | |
| | (g) | (g) | (g) | (g) | Average |
| Actual Yield | 78.83 | 83.39 | 85.65 | 85.3 | |
| Recovery % | 92.26 | 97.59 | 100.24 | 99.83 | 97.48 |

XRD and titration of as synthesised iron (II) oxalate were used to verify the formation of iron (II) oxalate. The XRD results exhibited the typical phase structure of ß-iron (II) oxalate and no other impurity phases were identified in the samples. The titration results also showed that the iron content in as synthesized iron (II) oxalate (31.15%) was close to its theoretical value (31.04%).

Notably, iron (II) oxalate synthesised using iron powder has slightly elevated levels of Ni (62 ppm vs 9 ppm) and V (132 ppm vs<5 ppm) impurity while the commercial iron (II) oxalate has higher levels of Mn (1260 ppm vs 92 ppm) and Zn (30 ppm vs<2 ppm) impurity. Ni and V impurity in as synthesised iron (II) oxalate comes from the iron powder (Ni as 73.8 ppm and V as 277 ppm) and is dependent on the purity of the iron raw materials.

### Example 2: Method of Producing Iron (II) Oxalate from Iron Oxides

To further confirm the feasibility of sulphuric acid re-utilisation for iron (II) oxalate synthesis, different sources of iron oxides were used as the iron source. The use of iron oxides as iron raw materials can further reduce the cost of iron (II) oxalate synthesis. Typically, iron oxides can be either naturally mined or produced synthetically. Two scenarios for the synthesis of iron (II) oxalate using iron oxides were evaluated: (a) using a synthetic pure source, and (b) using a naturally mined (or impure) iron source.

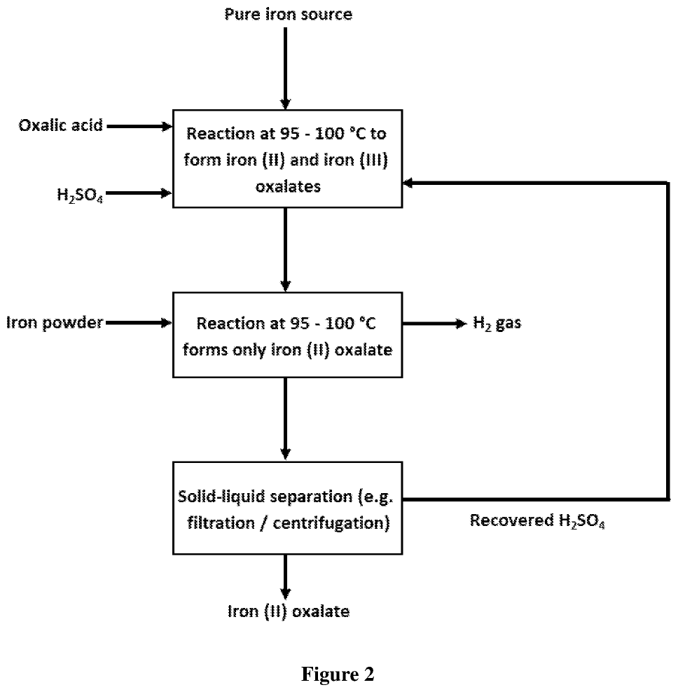

FIG. 2 is a process flow diagram illustrating an embodiment of the invention for the preparation of iron (II) oxalate using synthetic pure iron oxide. The first step is the digestion of iron oxide in a mixture of sulphuric acid and oxalic acid solution. During the digestion process, a small amount of iron powder can also be added to accelerate the digestion of certain iron oxides such as haematite. Usually, the digestion of iron oxide in the solution of sulphuric acid and oxalic acid produces a mix of iron (II) and iron (III) oxalates. Once the digestion of iron oxide is completed, a stoichiometric amount of iron powder (rapid rust or iron chips) is then added to reduce iron (III) oxalate to iron (II) oxalate.

The amount of iron powder added can be varied based on the excess of oxalic acid added in the digestion step. In this case, a 2-fold molar excess of oxalic acid to Fe content in iron oxide was added for the digestion process. The iron powder thereby utilises the excess oxalic acid in the solution to form iron (II) oxalate. Iron (II) oxalate product can then be recovered via simple filtration or centrifugation. This approach regenerates the sulphuric acid which can be reused for another cycle of iron (II) oxalate synthesis, reducing the chemical cost for synthesis of iron (II) oxalate.

To verify the technical feasibility of using iron oxide as raw materials for iron (II) oxalate synthesis, synthetic iron oxides (i.e. magnetite and haematite, as received from Lanxess AG, Germany) were used. Two cycles of iron (II) oxalate were prepared to illustrate the reusability of waste sulphuric acid. Table 2 below shows the percentage recovery of iron (II) oxalate using synthetic magnetite and synthetic haematite. The average recovery of iron (II) oxalate after two cycles is similar for both forms of iron oxides and is also similar to that recovered using pure iron powder.

TABLE 2
Yield of iron (II) oxalate at different cycles using
synthetic haematite and magnetite as an iron source
to illustrate sulphuric acid regeneration

| | | | Theoretical yield (g) | | |
|---|---|---|---|---|---|
| | | | 128.17 | | |
| | | | 1st cycle | 2nd cycle | |
| | | | (g) | (g) | Average |
| 1 | Lanxess | Actual Yield | 117.1 | 124.75 | |
| | Haematite | Recovery % | 91.36 | 97.33 | 94.35 |
| | | | Theoretical yield (g) | | |
| | | | 170.90 | | |
| | | | 1st cycle | 2nd cycle | |
| | | | (g) | (g) | Average |
| 2 | Lanxess | Actual Yield | 141.83 | 177.18 | |
| | Magnetite | Recovery % | 82.99 | 103.68 | 93.33 |

XRD spectra confirmed the formation of iron (II) oxalate and demonstrated that the iron (II) oxalate can be synthesised using this approach. The titration results and ICP analysis showed that the Fe content in iron (II) oxalates synthesised using synthetic iron oxides were equivalent to commercial samples. Furthermore, ICP analysis showed that the purity of iron (II) oxalate prepared using synthetic iron oxide was s equivalent to the commercial iron (II) oxalate.

The levels of trace impurities in iron (II) oxalate are dependent on the purity of synthetic iron oxides and iron powder used. For example, the levels of Cr (37 ppm for Magnetite, 60 ppm for Haematite and <10 ppm for commercial sample) and Ni (69.4 ppm for Magnetite, 59.2 ppm for Haematite and 9 ppm for commercial samples) impurities are slightly higher in iron (II) oxalate prepared using synthetic iron oxide, while that of Mn is higher in commercial iron (II) oxalate (158 ppm for Magnetite, 41 ppm for Haematite and 1260 ppm for the commercial sample).

### Example 3: Method of Producing Iron (II) Oxalate from Naturally Mined or Impure Iron Oxides

Naturally mined iron oxides are the cheapest form of iron and thus present an interesting choice for the synthesis of iron (II) oxalate. Naturally mined iron oxides, however, have a range of impurities such as silicates, aluminium oxides, titanium oxides etc. which need to be removed to provide a high-quality iron (II) oxalate product.

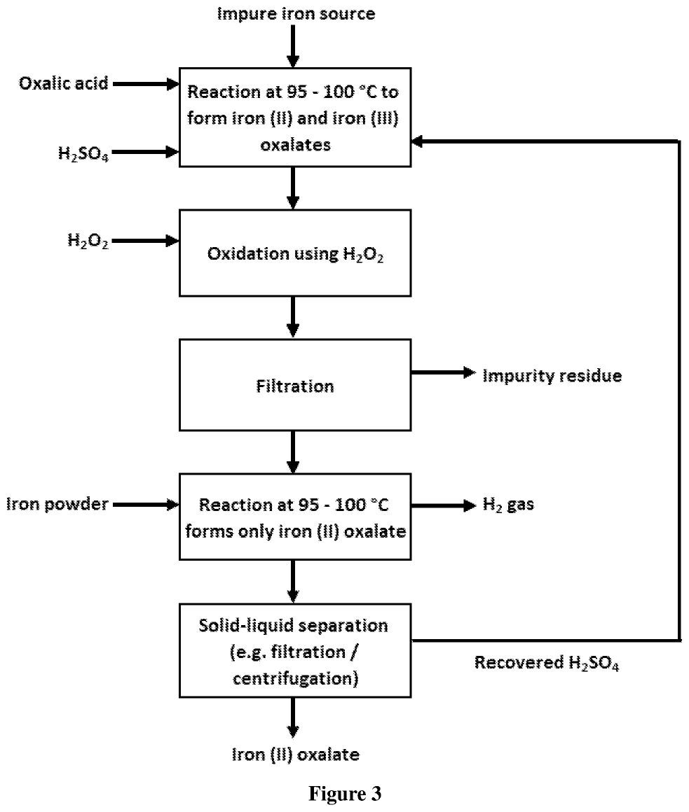

FIG. 3 is a process flow diagram illustrating an embodiment of the invention for the preparation of iron (II) oxalate using naturally mined iron oxides. The synthesis of iron (II) oxalate using natural (or mine grade) iron oxides follows similar steps to that using synthetic (or pure) iron oxide except with an additional purification step to remove the impurities. Briefly, the digestion of natural iron oxide produces a mixture of iron (II) and iron (III) oxalates. After the digestion, iron (II) oxalates in the solution can be oxidised using hydrogen peroxide to form water-soluble iron (III) oxalates. The oxidised iron (III) oxalate can then be filtered to remove insoluble metal oxalate impurities formed during the reaction (1st purification). Iron powder can then be added to the filtered iron (III) oxalate to form insoluble iron (II) oxalate. Finally, a relatively pure iron (II) oxalate can be recovered via filtration or centrifugation (2nd purification). The waste sulphuric acid can be recycled for the production of subsequent iron (II) oxalate.

In this example, three natural (mineral) iron source materials were investigated: (a) iron carbonate, (b) magnetite, and (c) haematite.

### (a) Iron Carbonate

Natural iron carbonate has a range of impurities such as aluminium oxide, silica, calcium oxides, titanium oxides etc. A natural iron carbonate with an approximate iron content of 36-38% was used for synthesis of iron (II) oxalate.

Si, Al, Ca, Mg are the major impurities in iron carbonate. However, these impurities are insoluble during digestion and can be removed in the 1^{st} purification step. A relatively pure iron (II) oxalate was produced and obtained after the 2^{nd} purification step (see Table 3 below which shows the level of impurities obtained from ICP analysis in the original FeCO₃ sample, in the filter residue after the first purification step, and in the iron (II) oxalate product).

TABLE 3
ICP analysis showing impurity levels in feed FeCO₃, filter residue
after 1st purification step, and in the iron (II) oxalate product.

| | Wt % present | Wt % present in | Wt % present in iron |
|---|---|---|---|
| Impurity | in feed FeCO₃ | filter residue | (II) oxalate product |
| SiO₂ | 12.75 | 64.4 | <0.01 |
| Al₂O₃ | 4.56 | 18.15 | 0.05 |
| CaO | 1.06 | 0.04 | 0.02 |
| MgO | 1.71 | 0.37 | 0.08 |
| Na₂O | 0.02 | 0.08 | <0.01 |
| K₂O | 0.35 | 1.43 | <0.01 |
| Cr₂O₃ | 0.003 | 0.008 | <0.002 |
| TiO₂ | 0.18 | 0.9 | 0.01 |
| MnO | 0.51 | 0.01 | 0.06 |
| P₂O₅ | 0.04 | 0.11 | <0.01 |
| SrO | 0.02 | 0.07 | <0.01 |
| BaO | 0.02 | 0.11 | <0.01 |

XRD spectra and titration for Fe content also confirmed the formation of iron (II) oxalate without significant impurities. Furthermore, the levels of trace impurities in iron (II) oxalate produced using natural iron carbonate is similar to commercial iron (II) oxalate (except Ni, 67.5 ppm and Co, 55.7 ppm that were introduced mainly from iron powder during 2nd stage purification). This demonstrates that the 2-step purification can remove impurities present in iron carbonate and a relatively pure iron (II) oxalate can be produced using natural iron carbonate.

### (b) Natural Magnetite

To further validate the two-step purification process for iron (II) oxalate synthesis using naturally mined iron oxides, iron (II) oxalate was also prepared using natural magnetite. A naturally mined magnetite with a specification of 68-70% Fe content from Anglo Pacific, UK was used for the synthesis.

Al, Ca, Mg, Si and Ti were the major impurities in the magnetite. After the two-stage purification step, most of these impurities were removed from the iron (II) oxalate. Furthermore, the trace level impurities in the product iron (II) oxalate was found to be equivalent to or lower than that of commercial iron (II) oxalate. The product iron (II) oxalate has slightly higher Ni (42.5 ppm) content than commercial iron (II) oxalate (9 ppm). The source of Ni originates from the iron powder and from the magnetite itself. A lower impurity iron powder and/or magnetite can further reduce the impurity in iron (II) oxalate. Other impurities such as Cr, Co, Mn, and Zn were found to be lower than in the commercial iron (II) oxalate.

The influence of the 1^{st} purification step (i.e. hydrogen peroxide addition) in the impurity removal from iron (II) oxalate was also explored. Iron (II) oxalate prepared without the 1st purification step has significantly lower levels of Al, Ca, Mg and Ti impurities as compared to the starting magnetite material. The results are shown in Table 4 below.

TABLE 4
ICP analysis showing impurity levels in feed magnetite
and in the iron (II) oxalate product (a) without 1^{st} purification
step and (b) including 1^{st} purification step.

| | | Wt % present in | Wt % present in |
|---|---|---|---|
| | | iron (II) oxalate | iron (II) oxalate |
| | Wt % present in | product without 1^{st} | product including 1^{st} |
| Impurity | feed magnetite | purification step | purification step |
| SiO₂ | 0.65 | 0.37 | <0.01 |
| Al₂O₃ | 0.25 | 0.07 | <0.01 |
| CaO | 0.28 | 0.06 | 0.01 |
| MgO | 0.35 | 0.05 | 0.01 |
| Na₂O | 0.03 | <0.01 | <0.01 |
| K₂O | 0.02 | 0.01 | 0.02 |
| Cr₂O₃ | 0.006 | 0.002 | <0.002 |
| TiO₂ | 0.36 | 0.02 | 0.02 |
| MnO | 0.06 | 0.06 | 0.01 |
| P₂O₅ | 0.05 | <0.01 | <0.01 |
| SrO | 0.01 | <0.01 | <0.01 |
| BaO | <0.01 | <0.01 | <0.01 |

The results show that synthesis of the iron (II) oxalate without the 1st purification step can remove a certain portion of the impurities. However, the levels of the impurities are still relatively high, and insoluble impurities such as Si may not be satisfactorily removed without a 2-step purification process.

### (c) Natural Haematite

The applicability of two-stage purification for the synthesis of iron (II) oxalate was also explored for low purity haematite. A 96% pure haematite from Chem Supply (CS) was used for the study.

Si, Al and Ca are the major impurities in the Haematite. Similar to other iron oxides, a two-step purification can remove the majority of these impurities and the as synthesised iron (II) oxalate has equivalent purity to commercial iron (II) oxalate. The results are shown in Table 5 below. Furthermore, using higher-grade iron chips for the second purification step significantly reduced the trace levels of Cr, Co, Ni and Mn impurities from the iron (II) oxalate.

TABLE 5
ICP analysis showing impurity levels in feed haematite
and in the iron (II) oxalate product

| | Wt % present in | Wt % present in iron |
|---|---|---|
| Impurity | feed haematite | (II) oxalate product |
| SiO₂ | 1.92 | <0.01 |
| Al₂O₃ | 2.12 | 0.04 |
| CaO | 0.29 | <0.01 |
| MgO | 0.05 | <0.01 |
| Na₂O | <0.01 | <0.01 |
| K₂O | <0.01 | <0.01 |
| Cr₂O₃ | 0.008 | 0.002 |
| TiO₂ | 0.04 | <0.01 |
| MnO | 0.03 | <0.01 |
| P₂O₅ | 0.07 | <0.01 |
| SrO | 0.01 | 0.01 |
| BaO | <0.01 | <0.01 |

### Example 4: Production of Iron (II) Oxalate Using Iron Source from Lanthanide Mine Tailings

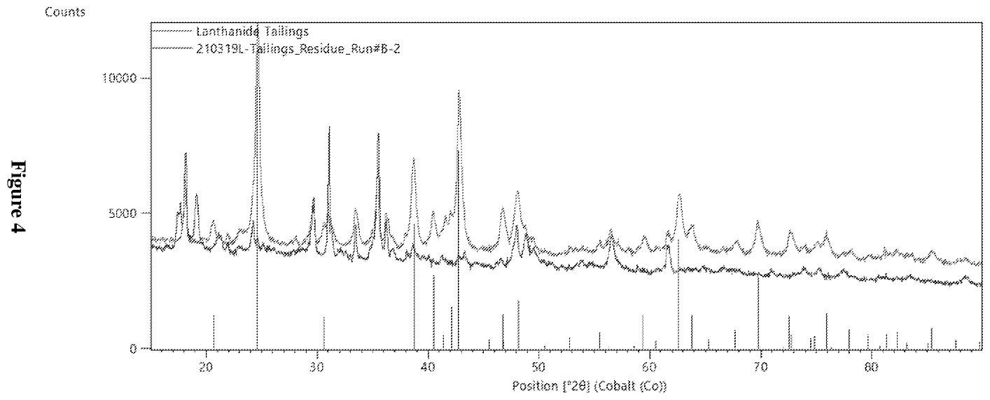

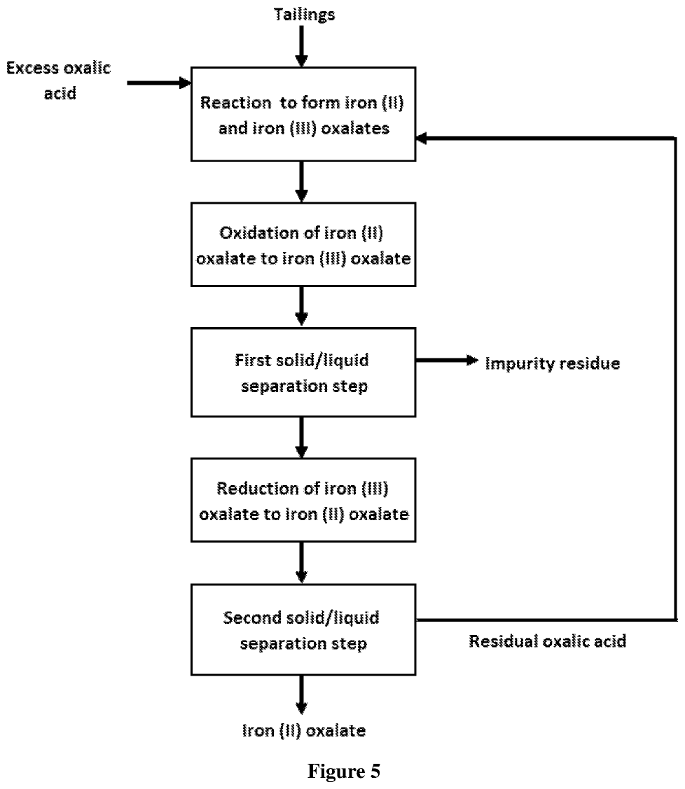

Mine tailings from lanthanide production was trialed for the synthesis of iron (II) oxalate. XRD in FIG. 4 shows that the Lanthanide tailing consisted of mainly goethite and other possible rare earth elements as its components. The tailings were subjected to the two-step purification process for the synthesis of iron (II) oxalate as generally illustrated in FIG. 5. Here, the digestion liquor consisted of only oxalic acid. The molar ratio of oxalic acid to the tailings was 2.1:1. The exclusion of the sulphuric acid from the digestion liquor slowed the digestion period from 4 hr to approximately 10 hrs. However, similar mass of the tailings (˜70%) could be digested and the undigested residues were removed during the first purification step after hydrogen peroxide addition. The XRD in FIG. 4 shows that most of the goethite from the tailings can be digested and the remaining residue contained other impurities from the tailings. The digested goethite was then converted into iron (II) oxalate during the second purification step via the addition of iron powder. The iron (II) oxalate was recovered via filtration and the digestion liquor was recycled for the next cycle of digestion.

Although the invention has been described in connection with preferred embodiments thereof, it should be understood that various modifications, additions and alterations may be made to the invention by one skilled in the art without departing from the spirit and scope of the invention as defined in the appended claims.

Furthermore, whilst features of the subject matter are described as particular aspects or embodiments or forms thereof, various combinations of these are contemplated. That is, all potential combinations of features that do not detrimentally affect the methods or processes described herein are contemplated.

1. A method for producing iron (II) oxalate from an insoluble iron material, the method comprising:
digesting the insoluble iron material with a digestion liquor comprising one or more acids, and reacting digested iron species with a source of oxalate, the oxalate being in stoichiometric excess relative to the digested iron species, to form a slurry comprising iron (II) oxalate and residual oxalate;
separating the iron (II) oxalate from the slurry to provide a solids stream comprising the iron (II) oxalate and a liquids stream comprising residual oxalate; and
recycling the liquids stream as the source of oxalate or a component thereof.

2. The method of any one of the preceding claims, wherein the digestion liquor comprises an aqueous solution of oxalic acid, the oxalic acid providing the source of oxalate.

3. The method of any one of the preceding claims, wherein the digestion liquor further comprises a mineral acid.

4. The method of any one of claim 3, wherein:
the digesting step comprises:
contacting the insoluble iron material with the digestion liquor, the digestion liquor comprising the mineral acid, and digesting the insoluble iron material to form a reaction solution comprising a soluble iron compound; and
reacting the soluble iron compound in the reaction solution with oxalic acid to form a slurry comprising iron (II) oxalate, residual oxalic acid, and regenerated mineral acid;
the separating step comprises:
separating the iron (II) oxalate from the slurry to provide a solids stream comprising the iron (II) oxalate and a liquids stream comprising residual oxalic acid and the regenerated mineral acid; and
the recycling step comprises:
recycling the liquids stream as digestion liquor.

5. The method of any one of the preceding claims, wherein the insoluble iron feed material comprises iron metal and/or synthetically produced iron compounds.

6. The method of any one of claims 1 to 4, wherein the insoluble iron feed material comprises: iron containing mineral tailings and/or iron containing waste and/or an iron ore and/or an iron ore concentrate.

7. The method of claim 5 or 6, wherein prior to the step of separating the iron (II) oxalate from the slurry to provide the solids stream and the liquids stream, the method further comprises:
oxidising iron (II) oxalate in the slurry to form a reaction solution comprising soluble iron (III) oxalate and solid impurities;
removing solid impurities from the reaction solution; and
reducing the iron (III) oxalate and forming a slurry comprising precipitated iron (II) oxalate.

8. A method for producing iron (II) oxalate from an iron material comprising impurities, the method comprising:
digesting the insoluble iron material with a digestion liquor comprising one or more acids, and reacting digested iron species with a source of oxalate, the oxalate being in stoichiometric excess relative to the digested iron species, to form a slurry comprising iron (II) oxalate, residual oxalate, and solid impurities;
oxidizing the iron (II) oxalate in the slurry to form a reaction solution, the reaction solution comprising soluble iron (III) oxalate, residual oxalate, and solid impurities;
separating the solid impurities from the reaction solution;
reducing the soluble iron (III) oxalate and forming a slurry comprising iron (II) oxalate and residual oxalate;
separating the iron (II) oxalate from the slurry to provide a solids stream comprising the iron (II) oxalate and a liquid stream comprising residual oxalate; and
recycling the liquids stream as the source of oxalate or a component thereof.

9. The method of claim 8, wherein the digestion liquor comprises an aqueous solution of oxalic acid, the oxalic acid providing the source of oxalate.

10. The method of claim 8 or 9, wherein the digestion liquor further comprises a mineral acid.

11. The method of claim 10, wherein:
the digesting step comprises:
contacting the insoluble iron material with the digestion liquor comprising the mineral acid and digesting the insoluble iron material to form a soluble iron compound;
reacting the soluble iron compound with oxalic acid to form the slurry comprising iron (II) oxalate, residual oxalic acid, regenerated mineral acid, and solid impurities;
the oxidizing step comprises:
oxidizing the iron (II) oxalate in the slurry to form the reaction solution, the reaction solution comprising soluble iron (III) oxalate, residual oxalic acid, regenerated mineral acid, and solid impurities;
the step of separating the solid impurities from the reaction solution comprises:
separating the solid impurities from the reaction solution;
the reducing step comprises:
reducing the soluble iron (III) oxalate and forming the slurry, the slurry comprising iron (II) oxalate, residual oxalic acid, and regenerated mineral acid;
the step of separating the iron (II) oxalate from the slurry comprises:
separating the iron (II) oxalate from the slurry to provide the solids stream comprising the iron (II) oxalate and the liquid stream comprising the residual oxalic acid and the regenerated mineral acid; and
the recycling step comprises:
recycling the recovered liquids stream as digestion liquor.

12. The method of any one of claims 8 to 11, wherein the insoluble iron feed material comprises: iron containing mineral tailings and/or iron containing waste and/or an iron ore and/or an iron ore concentrate.

13. The method of claim 12, wherein the iron ore and/or iron ore concentrate is selected from the group consisting of: haematite, magnetite, goethite, limonite, and/or siderite.

14. The method of claim any one of claims 7 to 13, wherein the step oxidizing the iron (II) oxalate includes reacting the iron (II) oxalate with hydrogen peroxide.

15. The method of any one of the preceding claims, wherein the insoluble iron feed material is digested at a temperature of from about 50° C. to about 100° C. and/or the soluble iron compound is reacted with a source of oxalate at a temperature of from about 50° C. to about 100° C.

16. The method of any one of the preceding claims, wherein the insoluble iron feed material is digested substantially under atmospheric pressure and/or the soluble iron compound is reacted with the source of oxalate substantially under atmospheric pressure.

17. The method of any one of the preceding claims, wherein the molar ratio of oxalic acid to iron in the insoluble iron feed material is at least 1.5 times the stoichiometric requirement.

18. A process for producing iron (II) oxalate, the process comprising:
providing a feed of an insoluble iron material into a reactor;
providing a feed of a digestion liquor comprising one or more acids into the reactor;
contacting the insoluble iron material with the digestion liquor in the reactor to digest the insoluble iron feed material;
reacting digested iron species in the reactor with a source of oxalate, the oxalate being in stoichiometric excess relative to the digested iron species, to form a slurry comprising iron (II) oxalate and residual oxalate;
subjecting the slurry to a solid-liquid separation process to provide a solids stream comprising iron (II) oxalate and a liquids stream comprising residual oxalate;
recycling the liquids stream into the reactor as the source of oxalate or a component thereof.

19. The process of claim 18, wherein the digestion liquor comprises a mineral acid and the source of oxalate is oxalic acid, and wherein:
the digesting step comprises:
contacting the insoluble iron material with the digestion liquor in the reactor to digest the insoluble iron material, the digestion liquor comprising a mineral acid to digest the insoluble iron material and form a reaction solution comprising a soluble iron compound;
the reacting step comprises:
reacting the soluble iron compound in the reaction solution with oxalic acid in the reaction vessel to form the slurry, the slurry comprising iron (II) oxalate, residual oxalic acid, and regenerated mineral acid;
subjecting the slurry to the solid-liquid separation process comprises:
subjecting the slurry to the solid-liquid separation process to provide the solids stream comprising the iron (II) oxalate and the liquids stream, the liquids stream comprising residual oxalic acid and the regenerated mineral acid; and
the recycling step comprises:
recycling the liquids stream comprising the regenerated mineral acid into the reactor as digestion liquor.

20. A process for producing iron (II) oxalate from an iron feed material comprising impurities, the process comprising:
providing a feed of an insoluble iron material comprising impurities into a reactor;
providing a feed of a digestion liquor comprising one or more acids into the reactor;
contacting the insoluble iron material with the digestion liquor in the reactor to digest the insoluble iron feed material;
reacting digested iron species in the reactor with a source of oxalate, the oxalate being in stoichiometric excess relative to the digested iron species, to form a slurry comprising iron (II) oxalate, residual oxalate, and solid impurities;
oxidizing the iron (II) oxalate in the slurry to form a reaction solution, the reaction solution comprising soluble iron (III) oxalate, residual oxalate, and solid impurities;
subjecting the reaction solution to a first solid-liquid separation process to separate the solid impurities from the reaction solution;
reducing the soluble iron (III) oxalate and forming a slurry comprising iron (II) oxalate and residual oxalate;
subjecting the slurry to a second solid-liquid separation process to provide a solids stream comprising iron (II) oxalate and a liquids stream comprising residual oxalate;
recycling the liquids stream into the reactor as the source of oxalate or a component thereof.

21. The process of claim 20, wherein the digestion liquor comprises a mineral acid and the source of oxalate is oxalic acid, and wherein:
the digesting step comprises:
contacting the insoluble iron material with the digestion liquor in the reactor to digest the insoluble iron material, the digestion liquor comprising a mineral acid to digest the insoluble iron material and form a reaction solution comprising a soluble iron compound;
the reacting step comprises:
reacting the soluble iron compound in the reaction solution with oxalic acid in the reactor to form the slurry, the slurry comprising iron (II) oxalate, residual oxalic acid, and regenerated mineral acid;
the oxidizing step comprises:
oxidising iron (II) oxalate in the slurry to form a reaction solution comprising soluble iron (III) oxalate and solid impurities
the step of subjecting the slurry to the first solid-liquid separation process comprises:
subjecting the reaction solution to the first solid-liquid separation process to separate the solid impurities from the reaction solution;
the step of reducing the soluble iron (III) oxalate comprises:
reducing the soluble iron (III) oxalate and forming the slurry comprising iron (II) oxalate, residual oxalic acid, and regenerated mineral acid;
the step of subjecting the slurry to the second solid-liquid separation process comprises:
subjecting the slurry to the second solid-liquid separation process to provide the solids stream comprising the iron (II) oxalate and the liquids stream, the liquid stream comprising residual oxalic acid and regenerated mineral acid;
the recycling step comprises:
recycling the liquids stream comprising the residual oxalic acid and the regenerated mineral acid into the reactor as digestion liquor.

22. An iron (II) oxalate product formed according to the method of any one of claims 1 to 18 or the process of any one of claims 18 to 21.

23. The iron (II) oxalate product of claim 22, wherein the iron (II) oxalate product has an Fe content of at least 29.7 wt %.
